(19) United States
(12) Patent Application Publication
(10) Pub. No.: US 2021/0324092 A1
(43) Pub. Date: Oct. 21, 2021

(54) MONOCLONAL ANTIBODY COMPOSITION FOR TREATMENT OF PHILADELPHIA CHROMOSOME POSITIVE ACUTE LYMPHOBLASTIC LEUKEMIA

(71) Applicants: Hassan Jumaa-Weinacht, Blaubeuren (DE); Mouhannad Jumaa, San Jose, CA (US); Ameera Alsadeq, Ulm (DE)

(72) Inventors: Hassan Jumaa-Weinacht, Blaubeuren (DE); Mouhannad Jumaa, San Jose, CA (US); Ameera Alsadeq, Ulm (DE)

(21) Appl. No.: 17/222,761

(22) Filed: Apr. 5, 2021

Related U.S. Application Data
(63) Continuation of application No. PCT/US2019/055809, filed on Oct. 11, 2019.
(60) Provisional application No. 62/745,215, filed on Oct. 12, 2018.

(51) Int. Cl. C07K 16/28; A61K 47/68; A61P 35/02; A61K 45/06

(52) U.S. Cl. CPC C07K 16/2866; A61K 47/6803; A61K 2039/505; A61K 47/6849; A61K 45/06; A61P 35/02

(57) ABSTRACT

A mechanism of action, way of treatment, composition and related methods for using the way of treatment and composition for the treatment of Philadelphia chromosome positive acute lymphoblastic leukemia is provided. The composition generally comprises a anti-IL7R antibody “monoclonal antibody” and/or a fragment of the anti-IL7R antibody.

### FIELD

A therapeutic target and composition comprising a monoclonal antibody for treating acute lymphoblastic leukemia, mechanism of action, target and methods for making and administering such a composition are provided. More particularly, the monoclonal antibody is useful for treating Philadelphia Chromosome Positive Acute Lymphoblastic Leukemia, and a therapeutic composition comprising the monoclonal antibody and methods for making and administering such a composition are provided.

### BACKGROUND

Acute lymphoblastic leukemia (ALL) is the most common blood cancer and childhood cancer. It affects white blood cells in the blood, particularly the lymphocyte cells. One subtype of this cancer is Philadelphia chromosome positive acute lymphoblastic leukemia (Ph+ ALL). Ph+ ALL is less common in children. However, it is common in adult ALL. About 30% of ALL cases in adults are positive for the Philadelphia chromosome (Ph+) and express an oncogenic fusion kinase termed BCR-ABL that drives the malignant transformation of precursor B cells.

Tyrosine kinase inhibitors, such as ABL kinase inhibitors, have been used to treat patients with Ph+ ALL. Tyrosine kinase inhibitors (TKI), such as imatinib, in combination with chemotherapy, are the current standard of care for treatment of Ph+ ALL. However, the development or presence of kinase inhibitor-resistant clones results in relapse and often incurable outbreak of the leukemia disease. In addition, the response for such treatment or treatment combination is in general low.

Other approaches to treatment of Ph+ ALL are standard combination chemotherapy and myeloablative allogeneic hematopoietic stem cell transplantation (HSCT). Ph+ ALL responds to combination chemotherapy, although complete remission is significantly less likely after standard induction regimens than in Ph− ALL. Combined with a short remission duration, there is a median event-free survival of 8 months; prognosis is poor. Five-year overall survival rates of between 10-20% are typical when treatment is chemotherapy alone. For this reason, myeloablative allogeneic HSCT has been a prominent focus in studies of Ph+ ALL.

Following a first successful treatment with chemotherapy, HSCT is commonly prescribed for both children and adults who are sufficiently fit and have a well-matched donor. However, at a conservative estimate, approximately half of all patients receiving standard induction therapy without a TKI will never undergo transplantation, even if a donor is available. Relapsed resistant Ph+ ALL is the predominant event preventing transplantation. Furthermore, myeloablative allogeneic HSCT, while having a highly significant effect on relapse in ALL, remains a dangerous treatment and the high mortality rate is delicately balanced against the benefits of the graft-versus-leukemia effect.

The use of TKIs against other kinases such spleen tyrosine kinase (SYK) has been proposed for treatment of certain forms of leukemia. However, it is not yet clear whether these will have sufficient therapeutic benefit in the treatment of Ph+ ALL.

### BRIEF SUMMARY

Although BCR-ABL kinase activity is required for the development of chronic myeloid leukemia (CML) and an aggressive form of ALL, other kinases, particularly SRC kinases, are involved in the development of Ph+ ALL suggesting that targeting these kinase is a useful addition to induction therapy for Ph+ ALL. In addition, targeting these kinases is of great importance for treatment of imatinib-resistant mutations in Ph+ ALL. Interestingly, there is increasing evidence of imatinib-resistant mutations already at diagnosis. The disadvantage of TKIs is that they do not enable long-term remission or cure for patients with Ph+ ALL. One common practice is to use TKIs in combination with chemotherapy to achieve a rapid response to facilitate early allogeneic HSCT which is presently considered to offer the best anti-leukemic activity.

There exists an immediate need for a treatment for resistant acute lymphoblastic leukemia, particularly Philadelphia chromosome positive acute lymphoblastic leukemia (Ph+ ALL) that is resistant to ABL kinase inhibitors.

The various embodiments can overcome the limitations of the prior treatment methods by providing a novel mechanism of action and composition comprising an anti-IL7R antibody for the treatment of acute lymphoblastic leukemia.

In one or more embodiments, a mechanism of action by directly targeting the IL7R is used to treat Philadelphia chromosome positive acute lymphoblastic leukemia.

In one or more embodiments, a mechanism of action by directly targeting the IL7R and without the need of action of killer cells is used to treat Philadelphia chromosome positive acute lymphoblastic leukemia.

In one or more embodiments, a composition comprising an anti-IL7R antibody is used to treat Philadelphia chromosome positive acute lymphoblastic leukemia.

In one or more embodiments, a composition comprising fragments of an anti-IL7R antibody is used to treat Philadelphia chromosome positive acute lymphoblastic leukemia

In one or more embodiments, the dosage form comprises or consists essentially of anti-IL7r monoclonal antibody, amino acid, a tonicity agent, a buffer, a chelating agent, antioxidants, surfactants/polymer, cryo or lyo protector, preservative, and has a pH about 5.5-7.5.

In one or more embodiments, the dosage form suitable to deliver the active agent in its stable, and soluble effective form.

In one or more embodiments, the amino acid stabilizer can be, but is not limited to, arginine, glutamic acid, isoleucine.

In one or more embodiments, the tonicity agent can be, but is not limited to, NaCl, Mannitol, dextrose, or sucrose.

In one or more embodiments, the buffering agent can be, but is not limited to, citrate (acid/Na/K), Phosphate (di/mono), Tris, or histidine. HCl and NaOH can be for, e.g., pH adjustment.

In one or more embodiments, the chelating agent could be, but not limited, EDTA or DTPA.

In one or more embodiments, the antioxidants can be, but are not limited to, Methionine and/or Glutathione.

In one or more embodiments, the surfactant can be, but is not limited to, Polysorbate or Pluronic.

In one or more embodiments, the polymers can be, but are not limited to, PVP or PEG.

In one or more embodiments, the cryo or lyo protector can be, but are not limited to, Sucrose, Trehalose, Mannitol, or Sorbitol.

In one or more embodiments, the preservative can be, but is not limited to, Phenol, Benzyl alcohol, or M cresol.

Additional embodiments of the composition, related methods, components of the composition, and the like will be apparent from the following description, examples, figures and claims. These and other objects and features of the disclosure will become more fully apparent when read in conjunction with the following detailed description.

### BRIEF DESCRIPTION OF THE DRAWINGS

FIG. 1 depicts a graph showing the enrichment of pre-B cells transduced with BCR-ABL retroviral vector in the absence of growth factor. EV: Empty vector.

FIG. 2 depicts a graph showing the death of IL7R^{fl/fl} pre-B cells which were transformed with BCR-ABL due to the deletion of IL7R using tamoxifen (Tam) induced Cre expression system.

FIG. 3 depicts a graph containing luciferase bioimaging of NOD-SCID mice that were injected with IL7R^{fl/fl}-BCR-ABL containing either a Cre-ER^{T2} construct or an empty vector control (ER^{T2}).

FIG. 4 depicts a graph showing Kaplan-Meier survival curves for NOD-SCID mice which were injected with IL7R^{fl/fl}-BCR-ABL pre-B cells containing either ER^{T2} or Cre-ER^{T2} and which were treated with tamoxifen.

FIG. 5 depicts a graph showing upregulation of IL7R, CXCR4, CRLF2, γc and TSLP expression levels in response to treating BCR-ABL⁺-pre-B cells with 5 μM imatinib for 15 hours.

FIG. 6 depicts a graph showing the rescue of BCR-ABL+ pre-B cells from cell death induced by imatinib in the presence of IL7. Imatinib (1 μM), IL7 (2.5 ng/ml), CXCL12 (100 ng/ml), AMD3100 (10 μM).

FIG. 7 depicts a graph showing cell cycle analysis of BCR-ABL+ pre-B cells treated with imatinib in the presence of IL7.

FIG. 8 depicts a graph showing a Fab-Proximity Ligation Assay (PLA) analysis of IL7R-CXCR4 proximity in BM-derived B cells (n=317) or in mature splenic B cells (n=321). Close proximity is represented by red dots.

FIG. 9 depicts a graph showing that deletion of CXCR4 in CXCR4^{fl/fl} pre-B cells with tamoxifen (Tam) enhances B cell differentiation markers μ and kappa.

FIG. 10 depicts a graph showing calcium mobilization in WT or WT-BCR-ABL transformed pre-B cells after stimulation with 100 ng/ml CXCL12.

FIG. 11 depicts a colony formation assay for CXCR4^{fl/fl}-BCR-ABL pre-B cells after deletion of CXCR4 using Cre-ER^{T2} system.

FIG. 12 depicts a graph showing the percentage of living BCR-ABL+ cells after CXCR4 deletion using Cre-ER^{T2} system.

FIG. 13 depicts a graph of CXCL12-induced calcium flux after 48 hours of tamoxifen-induced CXCR4 deletion.

FIG. 14 depicts a graph of migration assay for BCR-ABL transformed pre-B cells toward CXCL12 (100 ng/ml) for 16 hours after deletion of either CXCR4 or IL7R.

FIG. 15 depicts a graph showing the survival curves of NSG mice injected with BCR-ABL+ human cell line SUP-B15 treated with either vehicle, imatinib, ruxolitinib or a combination of ruxolitinib and imatinib.

FIG. 16 depicts a graph showing the percentage of lymphoblast cells in the peripheral blood of NSG mice after application of imatinib (an ABL kinase inhibitor), anti-IL7R antibody, or a vehicle control at day 58 after injection.

FIG. 17 depicts a representative flow cytometry analysis of peripheral blood (PB), spleen and bone marrow (BM) infiltration by human leukemic blasts in control and treated animals at day 58 when all control and imatinib-treated mice were sacrificed due to appearance of leukemic symptoms.

FIG. 18 depicts a graph showing the corresponding survival rate for xenografted mice after treatment with anti-IL7R antibody, imatinib, or vehicle control.

FIG. 19 depicts a graph showing the effect of in vitro treatment of anti-IL7R antibody in inducing apoptosis of BCR-ABL⁺ xenograft cells. Apoptosis activates caspase-8 cleavage and leads to the release of the caspase-8 active fragment p18.

### DETAILED DESCRIPTION

The present invention will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art. As can be appreciated from the foregoing and following description, each and every feature described herein, and each and every combination of two or more of such features, is included within the scope of the present disclosure provided that the features included in that such combinations are not inconsistent. In addition, any feature or combination of features may be specifically excluded from any embodiment of the present invention. Additional aspects and advantages of the present invention are set forth in the following description and claims, particularly when considered in conjunction with the accompanying examples and drawings.

All publications, patents and patent applications cited herein are hereby incorporated by reference in their entirety, unless otherwise indicated. In an instance in which the same term is defined both in a publication, patent, or patent application incorporated herein by reference and in the present disclosure, the definition in the present disclosure represents the controlling definition. For publications, patents, and patent applications referenced for their description of a particular type of compound, chemistry, etc., portions pertaining to such compounds, chemistry, etc. are those portions of the document which are incorporated herein by reference.

It must be noted that, as used in this specification, the singular forms “a,” “an,” and “the” include plural referents unless the context clearly dictates otherwise. Thus, for example, reference to an “antibody” includes a single antibody, monoclonal antibody as well as two or more different antibodies, reference to an “antigen” refers to a single antigen as well as to two or more different antigens, and the like. Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

As used herein, by the phrase “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Where a range of values is provided, it is understood that the upper and lower limit, and each intervening value between the upper and lower limit of the range is encompassed within the embodiments, unless doing so would be inconsistent with the text or otherwise noted.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As noted above, the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, any appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the contents herein have been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

Any of the features of an embodiment is applicable to all other aspects and other embodiments identified herein. Moreover, any of the features of an embodiment is independently combinable, partly or wholly, with other embodiments described herein in any way, e.g., one, two, or three or more embodiments may be combinable in whole or in part. Further, any of the features of an embodiment may be made optional to other aspects or other embodiments. Any aspect or embodiment of a method may be performed using a composition of another aspect or embodiment, and any aspect or embodiment of a composition can be configured to be used in a method of another aspect or embodiment.

In describing the embodiments, the following terminology will be used in accordance with the definitions described below.

The term “IL7” as used herein is a broad term, and is to be given its ordinary and customary meaning to a person of ordinary skill in the art (and is not to be limited to a special or customized meaning), and refers without limitation to interleukin 7, a cytokine which is important for B and T cell development.

The term “IL7R” as used herein is a broad term, and is to be given its ordinary and customary meaning to a person of ordinary skill in the art (and is not to be limited to a special or customized meaning), and refers without limitation to interleukin 7 receptor. IL7R is a heterodimer surface protein and consists of two subunits, interleukin7 receptor a chain (CD127) and common-gamma chain (CD132). CD127 can also heterodimerize with CRLF2.

The term “antibody” as used herein is a broad term, and is to be given its ordinary and customary meaning to a person of ordinary skill in the art (and is not to be limited to a special or customized meaning), and refers without limitation to an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also, unless otherwise specified, any antigen binding portion thereof that competes with the intact antibody for specific binding, fusion proteins comprising an antigen binding portion, and any other modified configuration of the immunoglobulin molecule that comprises an antigen recognition site. Antigen binding portions include, for example, Fab, Fab′, F(ab′)2, Fd, Fv, domain antibodies (dAbs, e.g., shark and camelid antibodies), fragments including complementarity determining regions (CDRs), single chain variable fragment antibodies (scFv), maxibodies, minibodies, intrabodies, diabodies, triabodies, tetrabodies, v-NAR and bis-scFv, and polypeptides that contain at least a portion of an immunoglobulin that is sufficient to confer specific antigen binding to the polypeptide. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively.

The subunit structures and three-dimensional configurations of different classes of immunoglobulins are well known.

The term “pharmaceutically acceptable” as used herein is a broad term, and is to be given its ordinary and customary meaning to a person of ordinary skill in the art (and is not to be limited to a special or customized meaning), and refers without limitation to an entity or ingredient is one that may be included in the compositions provided herein and that causes no significant adverse toxicological effects in the patient at specified levels, or if levels are not specified, in levels known to be acceptable by those skilled in the art. All ingredients in the compositions described herein are provided at levels that are pharmaceutically acceptable. For clarity, active ingredients may cause one or more side effects and inclusion of the ingredients with a side effect profile that is acceptable from a regulatory perspective for such ingredients will be deemed to be “pharmaceutically acceptable” levels of those ingredients.

The term “therapeutically effective amount” as used herein is a broad term, and is to be given its ordinary and customary meaning to a person of ordinary skill in the art (and is not to be limited to a special or customized meaning), and refers without limitation to an amount of a pharmaceutical preparation, or amount of an active ingredient in the pharmaceutical preparation, that is needed to provide a desired level of active ingredient in the bloodstream or in a target tissue. The precise amount will depend upon numerous factors, e.g., the particular active ingredient, the components and physical characteristics of the pharmaceutical preparation, intended patient population, patient considerations, and the like, and can readily be determined by one skilled in the art, based upon the information provided herein and available in the relevant literature.

The term “patient” as used herein is a broad term, and is to be given its ordinary and customary meaning to a person of ordinary skill in the art (and is not to be limited to a special or customized meaning), and refers without limitation to a mammal suffering from or prone to a condition that can be prevented or treated by administration of a composition as provided herein, and includes both humans and animals.

The terms “optional” or “optionally” as used herein are broad terms, and are to be given their ordinary and customary meaning to a person of ordinary skill in the art (and is not to be limited to a special or customized meaning), and mean without limitation that the subsequently described circumstance may or may not occur, so that the description includes instances where the circumstance occurs and instances where it does not.

It is generally preferred to administer the compositions of the embodiments in an intravenous, subcutaneous, peritoneal, intramuscular, intraperitoneal, intradermal, or subcutaneous unit dosage form; however, other routes of administration are also contemplated. Contemplated routes of administration include but are not limited to oral, parenteral, intravenous, and subcutaneous. Unit dosage forms configured for administration once a day are particularly preferred; however, in certain embodiments it can be desirable to configure the unit dosage form for administration twice a day, or more.

The compositions of embodiments are preferably isotonic with the blood or other body fluid of the recipient. The isotonicity of the compositions can be attained using sodium tartrate, propylene glycol or other inorganic or organic solutes. Sodium chloride is particularly preferred. Buffering agents can be employed, such as acetic acid and salts, citric acid and salts, boric acid and salts, and phosphoric acid and salts. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. In certain embodiments it can be desirable to maintain the active compound in the reduced state. Accordingly, it can be desirable to include a reducing agent, such as vitamin C, vitamin E, or other reducing agents as are known in the pharmaceutical arts, in the formulation.

Viscosity of the compositions can be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the thickening agent selected. An amount is preferably used that will achieve the selected viscosity. Viscous compositions are normally prepared from solutions by the addition of such thickening agents.

A pharmaceutically acceptable preservative can be employed to increase the shelf life of the compositions. Benzyl alcohol can be suitable, although a variety of preservatives including, for example, parabens, thimerosal, chlorobutanol, or benzalkonium chloride can also be employed. A suitable concentration of the preservative is typically from about 0.02% to about 2% based on the total weight of the composition, although larger or smaller amounts can be desirable depending upon the agent selected. Reducing agents, as described above, can be advantageously used to maintain good shelf life of the formulation.

The monoclonal antibodies or biologics as described herein can be in admixture with a suitable carrier, diluent, or excipient such as sterile water, physiological saline, glucose, or the like, and can contain auxiliary substances such as wetting or emulsifying agents, pH buffering agents, gelling or viscosity enhancing additives, preservatives, flavoring agents, colors, and the like, depending upon the route of administration and the preparation desired. See, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18^{th} and 19^{th} editions (December 1985, and June 1990, respectively). Such preparations can include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. The presence of such additional components can influence the physical state, solubility, stability, rate of in vivo release, and rate of in vivo clearance, and are thus chosen according to the intended application, such that the characteristics of the carrier are tailored to the selected route of administration.

Preferably, a unit dosage form contains from about 10 mg or less to about 1,000 mg or more of a composition, more preferably from about 20, 30, 40, 50, 60, 70, 80, 90, or 100 mg to about 150, 200, 250, 300, 350, 400, 450, 500, 550, 600, 650, 700, 750, 800, or 900 mg. Most preferably, unit dosage forms are provided in a range of dosages to permit divided dosages to be administered. A dosage appropriate to the patient and the number of doses to be administered daily can thus be conveniently selected. In certain embodiments two or more therapeutics can be administered into a single dosage form (e.g., in a combination therapy); however, in other embodiments it can be preferred to provide the therapeutic agents in separate dosage forms.

Surfactants can also be employed, for example, anionic detergents such as sodium lauryl sulfate, dioctyl sodium sulfosuccinate and dioctyl sodium sulfonate, cationic such as benzalkonium chloride or benzethonium chloride, or nonionic detergents such as polyoxyethylene hydrogenated castor oil, glycerol monostearate, polysorbates, sucrose fatty acid ester, methyl cellulose, or carboxymethyl cellulose.

When administered in liquid form, a liquid carrier such as water, petroleum, oils of animal or plant origin such as peanut oil, mineral oil, soybean oil, or sesame oil, or synthetic oils can be added to the active ingredient(s). Physiological saline solution, dextrose, or other saccharide solution, or glycols such as ethylene glycol, propylene glycol, or polyethylene glycol are also suitable liquid carriers. The compositions can also be in the form of oil-in-water emulsions. The oily phase can be a vegetable oil, such as olive or arachis oil, a mineral oil such as liquid paraffin, or a mixture thereof. Suitable emulsifying agents include naturally-occurring gums such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate.

When a monoclonal antibody or biologic is administered by intravenous, parenteral, or other injection, it is preferably in the form of a pyrogen-free, parenterally acceptable aqueous solution or oleaginous suspension. Suspensions can be formulated according to methods well known in the art using suitable dispersing or wetting agents and suspending agents. The preparation of acceptable aqueous solutions with suitable pH, isotonicity, stability, and the like, is within the skill in the art. A preferred composition for injection preferably contains an isotonic vehicle such as 1,3-butanediol, water, isotonic sodium chloride solution, Ringer's solution, dextrose solution, dextrose and sodium chloride solution, lactated Ringer's solution, or other vehicles as are known in the art. In addition, sterile fixed oils can be employed conventionally as a solvent or suspending medium. For this purpose, any bland fixed oil can be employed including synthetic mono or diglycerides. In addition, fatty acids such as oleic acid can likewise be used in the formation of injectable preparations. The compositions can also contain stabilizers, preservatives, buffers, antioxidants, or other additives known to those of skill in the art.

The duration of the injection can be adjusted depending upon various factors, and can comprise a single injection administered over the course of a few seconds or less, to 0.5, 0.1, 0.25, 0.5, 0.75, 1, 2, 3, 4, 5, 6, 7, 8, 9, 10, 11, 12, 13, 14, 15, 16, 17, 18, 19, 20, 21, 22, 23, or 24 hours or more of continuous intravenous administration.

The compositions can additionally employ adjunct components conventionally found in pharmaceutical compositions in their art-established fashion and at their art-established levels. Thus, for example, the compositions can contain additional compatible pharmaceutically active materials for combination therapy (such as supplementary chemotherapeutics, antimicrobials, antipruritics, astringents, local anesthetics, anti-inflammatory agents, reducing agents, and the like), or can contain materials useful in physically formulating various dosage forms of the embodiments, such as excipients, dyes, thickening agents, stabilizers, preservatives or antioxidants.

The monoclonal antibodies or biologics of the embodiments can be provided to an administering physician or other health care professional in the form of a kit. The kit is a package which houses a container which contains the therapeutica agent in a suitable composition, and instructions for administering the composition to a subject. The kit can optionally also contain one or more additional therapeutic agents, e.g., chemotherapeutic agents. For example, a kit containing one or more compositions comprising compound(s) of the preferred embodiments in combination with one or more additional agents can be provided, or separate pharmaceutical compositions containing a compound of the preferred embodiments and additional therapeutic agents can be provided. The kit can also contain separate doses for serial or sequential administration. The kit can optionally contain one or more diagnostic tools and instructions for use. The kit can contain suitable delivery devices, e.g., syringes, and the like, along with instructions for administering the compound(s) and any other therapeutic agent. The kit can optionally contain instructions for storage, reconstitution (if applicable), and administration of any or all therapeutic agents included. The kits can include a plurality of containers reflecting the number of administrations to be given to a subject. In a particularly preferred embodiment, a kit is provided that includes the monoclonal antibody or biologic and another compound for the treatment of Philadelphia chromosome positive acute lymphoblastic leukemia.

### Overview

A mechanism of action to treat ALL by targeting IL7R is provided.

A mechanism of action to treat Philadelphia Chromosome Positive (Ph+) Acute Lymphoblastic Leukemia and imatinib-resistant Ph+ BCP-ALL by targeting IL7R is provided.

A mechanism of action that targets IL7R without the need of Natural Killer (NK) cells is provided.

TA composition and related methods for using the composition is provided. The composition generally comprises a monoclonal antibody for the treatment of Ph+ ALL. In exemplary embodiments, the monoclonal antibody is an anti-IL7R antibody.

Patients with ALL can be tested according to known tests to determine whether the type of ALL from which they suffer is Philadelphia chromosome positive or not. These tests of the cancer genes determine whether the particular type of cancer cells respond well to certain types of cancer-fighting drugs. Certain mutations, such as Philadelphia chromosome positive mutation, make ALL cells more difficult to fight with typical anti-cancer drugs and make patients more susceptible to relapse following treatment.

It has been discovered that initiation and maintenance of Ph+ ALL requires expression of the interleukin 7 receptor (IL7R) and that IL7R cooperates with the chemokine receptor CXCR4 to block precursor B cell differentiation and to activate BCR-ABL signaling thereby allowing deregulated cell proliferation and survival. The IL7/IL7R signaling provides a key resistance mechanism that limits the effectiveness of kinase inhibitors in inducing cell death in Ph+ ALL cells.

Treatments with tyrosine kinase inhibitors are ineffective in treatment of imatinib resistant Ph+ ALL because they fail to eliminate Ph+ ALL cells in vivo. The expression of IL7R and its downstream signaling machinery such as JAK1 and STAT5a are upregulated upon treatment with BCR-ABL kinase inhibitors. However, inhibition of IL7R signaling using available JAK kinase inhibitors failed to eliminate Ph+ cells in vivo. Efficient elimination of Ph+ ALL cells with resistance to imatinib, a commonly used ABL inhibitor, was achieved in vivo only after treatment with anti-IL7R antibody.

IL7R is required for the survival of Ph+ ALL cells. Treatment with anti-IL7R antibodies significantly reduces or even eliminates Ph+ ALL cells showing drug resistance. Anti-IL7R antibodies provide an effective treatment of ABL kinase inhibitor-resistant Ph+ ALL.

U.S. Patent Application 20170247460 by Geiger et Al., incorporated by reference herein in its entirety, describes anti-IL7R antibody compositions for treatment of autoimmune disorders, such as type 1 diabetes, multiple sclerosis, graft versus host disease, and lupus. Such compositions are examples of compositions that can be used in the treatment of Ph+ ALL. Other compositions will be evident to those skilled in the art.

### EXAMPLES

The following examples are put forth to provide those of ordinary skill in the art with a complete disclosure and description of how the target, the monoclonal antibody, the composition, its components, active ingredients, solvents, and the like, are prepared and evaluated, along with related methods, and are intended to be purely exemplary. Thus, the examples are in no way intended to limit the scope of what the inventors regard as their invention. There are numerous variations and combinations, e.g., component concentrations, desired solvents, solvent mixtures, antioxidants, and other mixture parameters and conditions that may be employed to optimize composition characteristics such as purity, yield, stability, odor, color, viscosity, penetration, and the like. Such are considered as well within the scope of the present disclosure.

### Example 1

### IL7R Expression is Required for Bcr-Abl Induced B Cell Precursor (Bcp−) Transformation and all Development

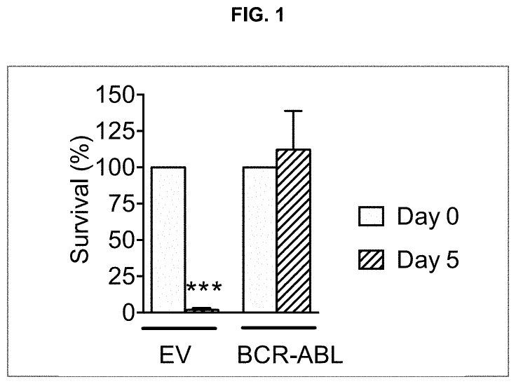

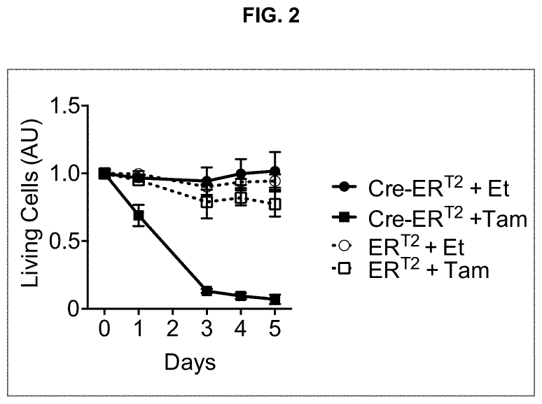

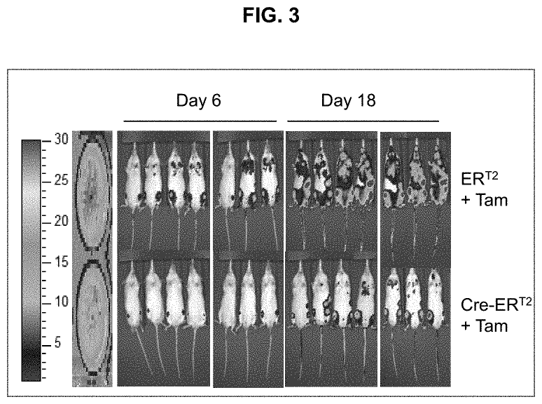

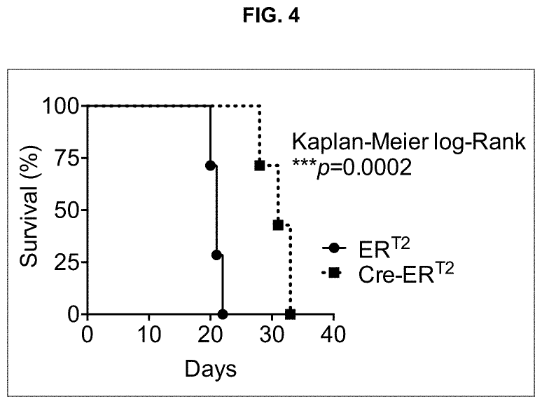

It has been discovered that IL7R expression is required for BCR-ABL induced precursor-B (pre-B) cell transformation and ALL development. This requirement was demonstrated by introducing a retroviral vector for BCR-ABL expression into bone marrow (BM)-derived pre-B cells from mice homozygous for IL7R loxP-flanked alleles (IL7^{fl/fl}). BCR-ABL expressing cells proliferated independent of IL7 which was used as growth factor in this in vitro culture (FIG. 1). For inducible deletion of IL7R^{fl/fl}, we introduced a tamoxifen-inducible Cre (Cre-ERT2) into the BCR-ABL-transformed cells. Inducible deletion of the IL7R gene led to cell death of the BCR-ABL-transformed ALL cells (FIG. 2). In addition, BCR-ABL-transformed cells induced leukemia development in mice within 3 weeks. IL7R deletion by tamoxifen treatment in vivo reduced leukemic cell burden and significantly prolonged mice survival compared to control mice (FIGS. 3 and 4). These data indicate that IL7R expression is required for initiation and maintenance of BCR-ABL-induced pre-B cell transformation.

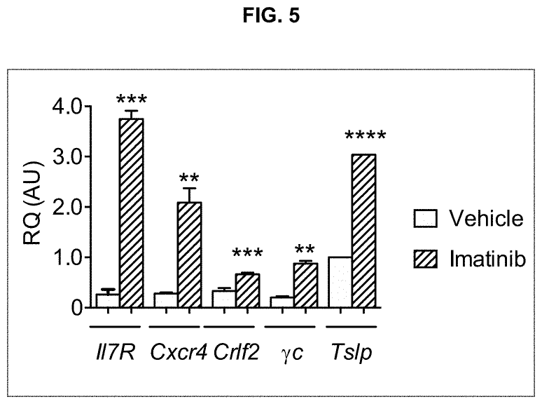

Imatinib treatment resulted in upregulation of IL7R expression together with the co-receptors CRLF2 and gamma chain, downstream signaling elements including JAK1 and STAT5a and the chemokine receptor CXCR4 (FIG. 5).

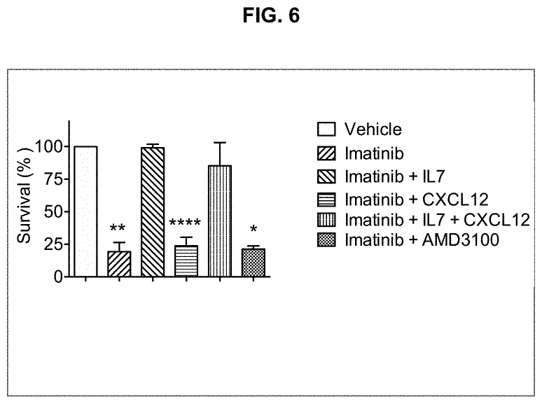

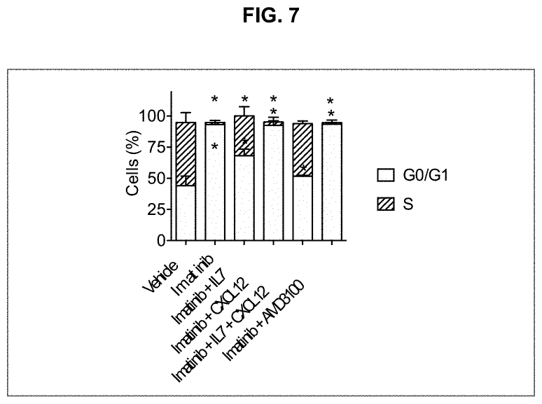

Treatment with IL7 counteracted the BCR-ABL inhibitor-induced cell death and restored cell cycling. Treatment with TSLP, the ligand for CRLF2, or with CXCL12, also known as SDF-1a the ligand for CXCR4 or its antagonist AMD3100 did not affect imatinib treatment (FIGS. 6 and 7).

### Example 2

### Bcr-Abl is Recruited into the Vicinity of Il7R to Activate Downstream Signaling Events Via Cxcr4

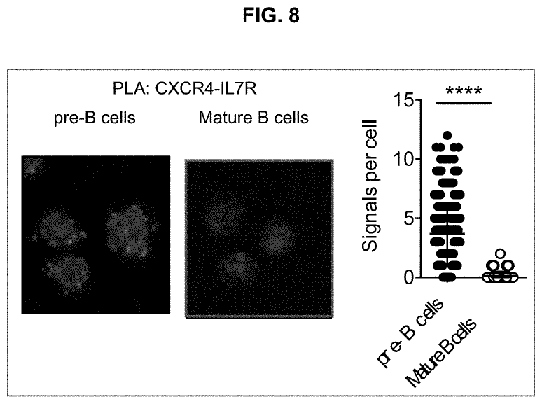

BCR-ABL is recruited into the vicinity of IL7R to activate downstream signaling events. BCR-ABL crosstalk with CXCR4, so the interaction between IL7R and CXCR4 was investigated by proximity ligation assay (PLA) as a mechanism to recruit BCR-ABL close to IL7R. Adjacent surface cell binding of the ligands IL7 (7 kD) and CXCL12 (15 kD), which both are smaller than antibody Fab fragment, suggested the corresponding receptors were localized at a proximity below 10 nm. Using directly labeled IL7 and CXCL12 it was revealed that IL7R and CXCR4 were localized in close proximity in precursor B cells (FIG. 8).

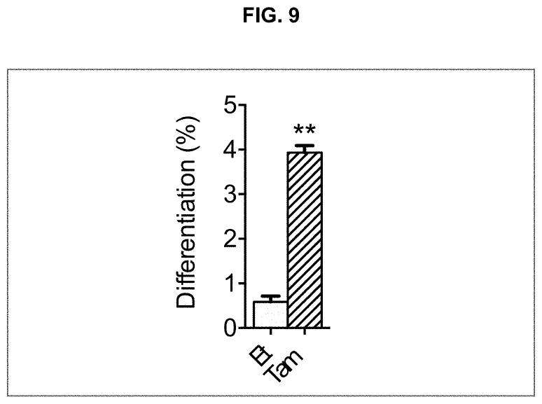

Since IL7R signaling affects precursor B cell differentiation, whether the association with CXCR4 synergized with IL7R signaling in blocking pre-B cell differentiation as measured by immunoglobulin light chain gene recombination was tested. BM-derived pre-B cell cultures from mice homozygous for loxP-flanked alleles CXCR4 (CXCR4^{fl/fl}) were established and Cre-ERT2 for inducible deletion of CXCR4 was introduced. The absence of CXCR4 lead to increased pre-B cells differentiation in the presence of IL7 suggesting that both receptors synergized to control the differentiation of early B cells (FIG. 9).

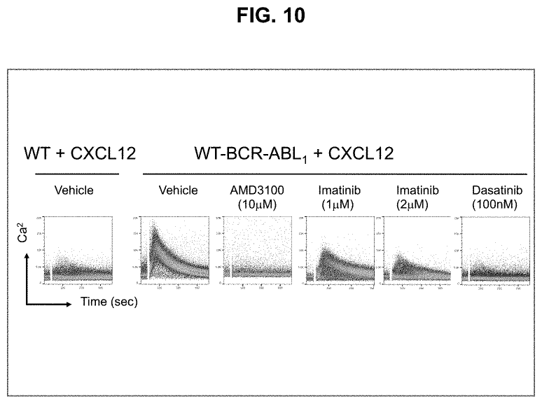

WT pre-B cells showed a negligible CXCL12-induced Ca2+ flux, the same cells showed a robust Ca2+ response after transformation with BCR-ABL. To show that the Ca2+ response requires binding of CXCL12 cells were treated with its antagonist AMD3100. Moreover, inhibiting BCR-ABL kinase activity via imatinib or dasatinib reduced the CXCL12-induced Ca2+ response and, most likely because of its effect on Src kinases, dasatinib showed an effective inhibition at much lower concentrations than imatinib (FIG. 10).

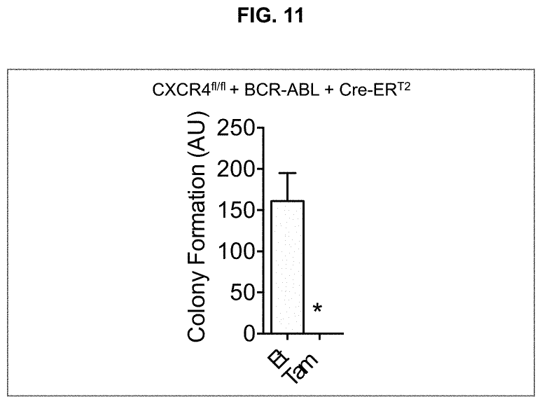

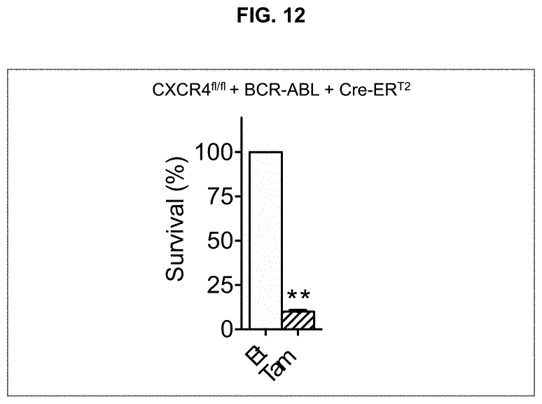

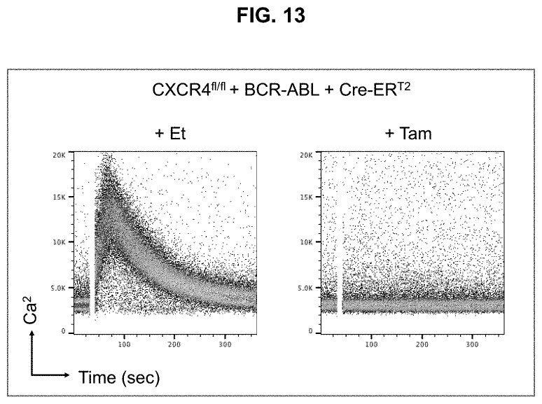

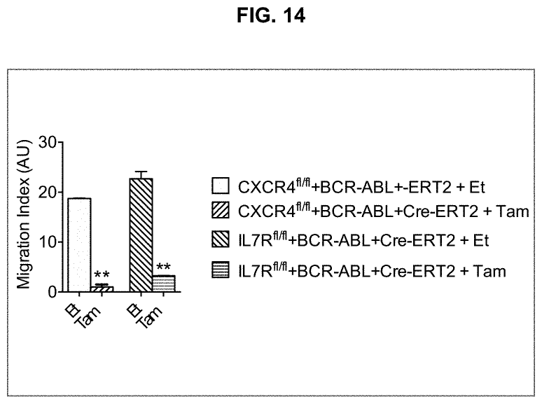

To further clarify the role of CXCR4 for BCR-ABL induced transformation, Cre-ERT2 was introduced into BCR-ABL transformed CXCR4^{fl/fl} cells. Tamoxifen-induced CXCR4 deletion blocked colony formation by BCR-ABL transformed cells and led to cell death within 3 days of in vitro culture (FIGS. 11 and 12). As expected, the CXCR4-deficient cells did not mobilize Ca2+ when treated with CXCL12 (FIG. 13). Both CXCR4-deficient and IL7R-defcient BCR-ABL-transformed cells showed an impaired migration toward CXCL12 gradient (FIG. 14).

### Example 3

### Specific Targeting of Il7R Using Monoclonal Antibody Reduces Leukemia Engraftment and Induces Cell Death in Bcr-Abl+ Human all Cells

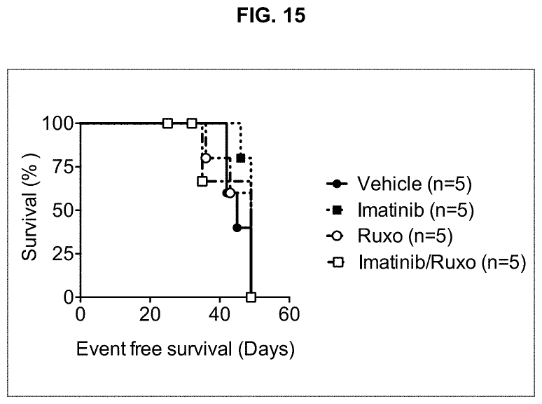

It was investigated whether inhibition of IL7R signaling using ruxolitinib, JAK1/JAK2 kinase inhibitor, can interfere with the survival of BCR-ABL transformed cells. Human BCR-ABL+ ALL cells were injected into immune deficient mice and the survival of recipient mice under imatinib only or combined imatinib/ruxolitinib treatment was monitored. The combination treatment was unable to prolong the survival of recipient mice or to reduce the percentage of leukemic cells in bone marrow (BM) and spleen (FIG. 15). These data indicated that ruxolitinib is not efficient for ALL treatment in vivo. This might be caused by different drug availability and insufficient inhibition of IL7R signaling as ruxolitinib mainly inhibited JAK1 and JAK2.

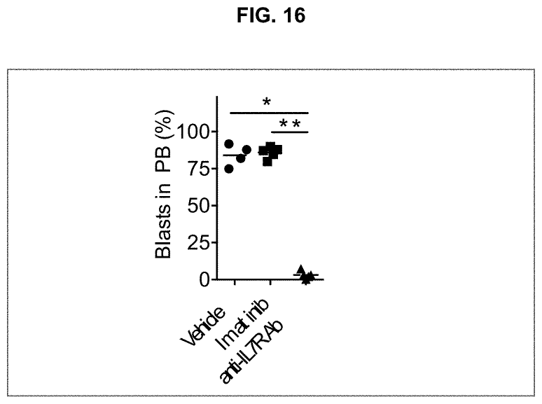

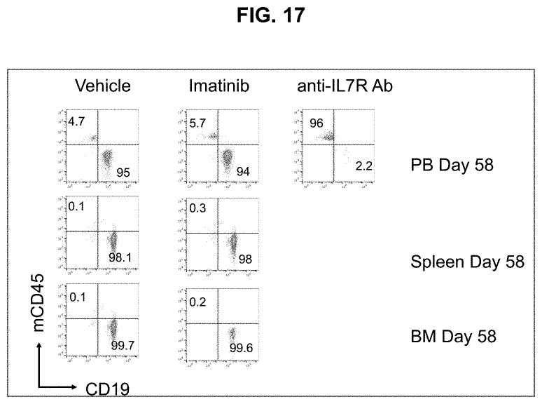

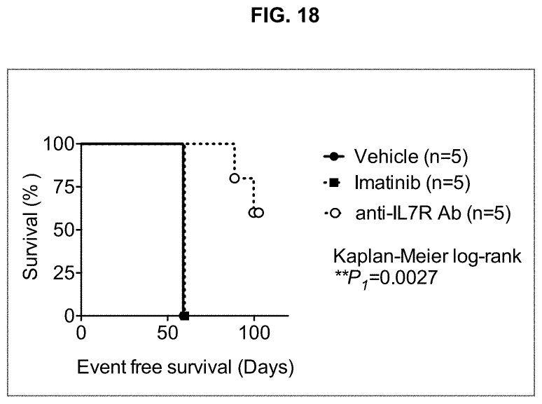

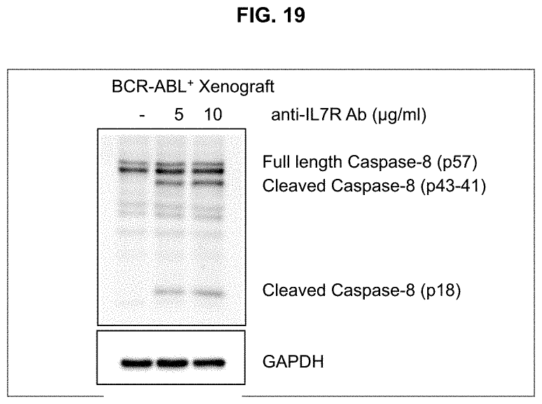

Blocking IL7R directly using specific monoclonal antibodies was tested in vivo. An imatinib-resistant BCR-ABL+ ALL patient cells were injected into immune deficient mice and treated them with antibodies against human IL7R. As expected due to kinase inhibitor resistance, imatinib was unable to prevent leukemia development and therefore, the leukemia burden and the survival of untreated mice were similar to imatinib treated mice (FIGS. 16 and 17). Importantly, anti-IL7R antibody significantly reduced leukemia infiltration and led to an expanded survival time (FIG. 18). In addition, IL7R antibody treatment in vitro induced apoptosis as shown by an increased cleavage of caspase-8 (FIG. 19). The finding that kinase inhibitor-resistant BCR-ABL+ BCP-ALL cells express IL7R and can be eliminated with anti-IL7R antibodies indicated that anti-IL-7R antibodies can be used effectively in ALL therapy and drug resistant ALL.

The methods of and compositions of the embodiments can be adapted to other anti-IL-7R antibodies, e.g., those undergoing clinic testing. Examples include GSK2618960 for pSS and Relapsing Remitting Multiple Sclerosis (RRMS), PF-06342674 (RN168) for MS, OSE-703 for solid tumors, and others as are known in the art.

The following heavy chain sequence is disclosed, where T can be A, H can be N, and I/L as in GL:

| | | |
|---|---|---|
| 001 | QVQLKESGPG LVAPSQSLSI TCTVSGFSLT SYGVHWVRQP PGKGLEWLGV | 050 |
| 051 | IWTGGSTHYN SALMSRLSIS KDNSKSQVFL KMNSLQTDDT AMYYCAREGD | 100 |
| 101 | YYASFAYWGQ GTLVTVSA. | |

The following light chain sequence is disclosed:

| | | |
|---|---|---|
| 001 | DIQMNQSPSS LSASLGDTIT ITCHASQNIN VWLSWYQQKP GNIPKLLIYK | 050 |
| 051 | ASNLHTGVPS RFSGSGSGTG FTLTISSLQP EDIATYYCQQ GQSYPYTFGG | 100 |
| 101 | GTKLEIKR | |

### Exemplary Compositions and Methods

Composition 1: A composition comprising a monoclonal antibody or biologic configured to bind to the IL7R, for use in the treatment of acute lymphoblastic leukemia.

Composition 2: Composition 1, for use in the treatment of Philadelphia chromosome positive acute lymphoblastic leukemia.

Composition 3: Composition 1, for use in the treatment of Resistant Philadelphia chromosome positive acute lymphoblastic leukemia.

Composition 4: Composition 1, comprising a monoclonal antibody.

Composition 5: Composition 1, wherein the monoclonal antibody or biologic is configured to bind to the IL7R common gamma chain.

Composition 6: Composition 1, wherein the monoclonal antibody is a fragment of anti-IL7R antibody.

Composition 7: Composition 1, wherein the monoclonal antibody is selected from the group consisting of murine, human, or humanized monoclonal antibody, and wherein the antibody is configured to bind to human IL-7R.

Composition 8: Composition 1, wherein the monoclonal antibody is only the antibody or conjugate to the antibody or conjugate to a fragment of anti-IL7R antibody.

Composition 9: Composition 1, wherein the monoclonal antibody comprises human framework regions, optionally for use in CAR-T-Cell immunotherapy.

Composition 10: Composition 1, wherein the monoclonal antibody comprises at least one human constant region.

Composition 11: Composition 10, wherein the at least one human constant region comprises a modification that increases binding to the Philadelphia chromosome positive in IL7R.

Composition 12: Composition 1, wherein the monoclonal antibody inhibits IL7 signaling in IL7R Philadelphia chromosome positive cells.

Composition 13: Composition 1, wherein the monoclonal antibody or biologics specifically bind to or target the extracellular domain of the IL7R.

Composition 14: Composition 1, wherein the monoclonal antibody comprises an anti-IL7R antibody that shows selectivity in targeting Philadelphia chromosome-positive IL7R antibodies relative to Philadelphia chromosome-negative IL7R antibodies, optionally for use CAR-T-Cell immunotherapy.

Composition 15: An antibody therapeutics composition adapted for administration by injection, comprising a therapeutically effective amount of the monoclonal antibody or biologics of any of Compositions 1-14 and a pharmaceutically acceptable excipient.

Composition 16: Composition 15, further comprising an effector molecule or a detectable marker wherein the monoclonal antibody or antigen binding fragment is linked to the effector molecule or the detectable marker.

Composition 17: Composition 15, further comprising a therapeutic drug, wherein the therapeutic drug and the monoclonal antibody form an antibody-drug conjugate.

Composition 18: Composition 17, wherein the therapeutic drug is a chemotherapeutic agent.

Composition 19: Composition 18, wherein the chemotherapeutic agent is a known chemotherapeutic agent for the treatment of acute lymphoblastic leukemia or chronic lymphocytic leukemia.

Composition 20: Composition 17, further comprising a cleavable peptide linker molecule, wherein the therapeutic drug is conjugated to the monoclonal antibody by the cleavable peptide linker.

Method 21: A method for treatment of acute lymphoblastic leukemia comprising: administering a therapeutically effective amount of the monoclonal antibody or biologics of any of claims 1-14 to a patient in need thereof for the treatment of Philadelphia chromosome positive acute lymphoblastic leukemia.

Method 22: Method 21, wherein the Philadelphia chromosome positive acute lymphoblastic leukemia is resistant to treatment with ABL kinase inhibitors.

Method 23: Method 21, wherein the administering step is repeated.

Method 24: Method 21, wherein the composition is in a form of a pharmaceutical formulation which keeps the monoclonal antibody stable and bioactive suitable for a possible route of administration including parenteral administration, including intravenous, intramuscular, intraperitoneal, intradermal, or subcutaneous.

Method 25: Method 21, wherein the composition comprises an anti-IL7r monoclonal antibody, an amino acid, a tonicity agent, a buffer, a chelating agent, antioxidant, surfactant/polymer, bulking agent, Cryo/lyo protector, and preservative.

Any individual feature of any exemplary method or composition is independently combinable, in whole or in part, with any other method or composition. Any exemplary method can be performed using any exemplary composition.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2^{nd} ed., Cold Spring Harbor Laboratory Press, Cold Spring Harbor, N.Y. (1989) and Coligan et al. Current Protocols in Immunology (Current Protocols, Wiley Interscience (1994)), which are incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

The terms “isolated polynucleotide” and “isolated nucleic acid segment” as used herein shall mean a polynucleotide of genomic, cDNA, or synthetic origin or some combination thereof, which by virtue of its origin the “isolated polynucleotide” or “isolated nucleic acid segment” (1) is not associated with all or a portion of a polynucleotide in which the “isolated polynucleotide” or “isolated nucleic acid segment” is found in nature, (2) is operably linked to a polynucleotide which it is not linked to in nature, or (3) does not occur in nature as part of a larger sequence.

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g., free of murine proteins, (3) is expressed by a cell from a different species, or, (4) does not occur in nature.

The term “polypeptide” as used herein is a generic term to refer to native protein, fragments, or analogs of a polypeptide sequence. Hence, native protein, fragments, and analogs are species of the polypeptide genus.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “operably linked” as used herein refers to positions of components so described are in a relationship permitting them to function in their intended manner. A control sequence “operably linked” to a coding sequence is ligated in such a way that expression of the coding sequence is achieved under conditions compatible with the control sequences.

The term “control sequence” as used herein refers to polynucleotide sequences which are necessary to effect the expression and processing of coding sequences to which they are ligated. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

The term “oligonucleotide” referred to herein includes naturally occurring, and modified nucleotides linked together by naturally occurring, and non-naturally occurring oligonucleotide linkages. Oligonucleotides are a polynucleotide subset generally comprising a length of 200 bases or fewer. In one embodiment, oligonucleotides are 10 to 60 bases in length, such as but not limited to, 12, 13, 14, 15, 16, 17, 18, 19, or 20 to 40 bases in length. Oligonucleotides are usually single stranded, e.g., for probes; although oligonucleotides may be double stranded, e.g., for use in the construction of a gene mutant. Oligonucleotides of the invention can be either sense or antisense oligonucleotides.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phoshoraniladate, phosphoroamidate, and the like. See e.g., LaPlanche et al. Nucl. Acids Res. 14:9081 (1986); Stec et al. J. Am. Chem. Soc. 106:6077 (1984); Stein et al. Nucl. Acids Res. 16:3209 (1988); Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990), the disclosures of which are hereby incorporated by reference. An oligonucleotide can include a label for detection, if desired.

The term “selectively hybridize” referred to herein means to detectably and specifically bind. Polynucleotides, oligonucleotides and fragments thereof in accordance with the invention selectively hybridize to nucleic acid strands under hybridization and wash conditions that minimize appreciable amounts of detectable binding to nonspecific nucleic acids. High stringency conditions can be used to achieve selective hybridization conditions as known in the art and discussed herein. Generally, the nucleic acid sequence homology between the polynucleotides, oligonucleotides, and fragments of the invention and a nucleic acid sequence of interest will be at least 80%, and more typically with increasing homologies of at least 85%, 90%, 95%, 99%, and 100%. Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, pp. 101-110 (Volume 5, National Biomedical Research Foundation (1972)) and Supplement 2 to this volume, pp. 1-10. The two sequences or parts thereof are more preferably homologous if their amino acids are greater than or equal to 50% identical when optimally aligned using the ALIGN program. The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The following terms are used to describe the sequence relationships between two or more polynucleotide or amino acid sequences: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity. A “comparison window”, as used herein, refers to a conceptual segment of at least 18 contiguous nucleotide positions or 6 amino acids wherein a polynucleotide sequence or amino acid sequence may be compared to a reference sequence of at least 18 contiguous nucleotides or 6 amino acid sequences and wherein the portion of the polynucleotide sequence in the comparison window may comprise additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman Adv. Appl. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), Geneworks, or MacVector software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, such as at least 90 to 95 percent sequence identity, or at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As used herein, the twenty conventional amino acids and their abbreviations follow conventional usage. See Immunology—A Synthesis (2^{nd} Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as α-,α-disubstituted amino acids, N-alkyl amino acids, lactic acid, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, ó-N-methylarginine, and other similar amino acids and imino acids (e.g., 4-hydroxyproline). In the polypeptide notation used herein, the lefthand direction is the amino terminal direction and the righthand direction is the carboxy-terminal direction, in accordance with standard usage and convention.

Similarly, unless specified otherwise, the lefthand end of single-stranded polynucleotide sequences is the 5′ end; the lefthand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences”.

As applied to polypeptides, the term “substantial identity” means that two peptide sequences, when optimally aligned, such as by the programs GAP or BESTFIT using default gap weights, share at least 80 percent sequence identity, such as at least 90 percent sequence identity, or at least 95 percent sequence identity, or at least 99 percent sequence identity. Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, glutamic-aspartic, and asparagine-glutamine.

As discussed herein, minor variations in the amino acid sequences of antibodies or immunoglobulin molecules are contemplated as being encompassed by the present invention, providing that the variations in the amino acid sequence maintain at least 75%, such as at least 80%, 90%, 95%, and 99%. In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the invention.

Preferred amino acid substitutions are those which: (1) reduce susceptibility to proteolysis, (2) reduce susceptibility to oxidation, (3) alter binding affinity for forming protein complexes, (4) alter binding affinities, and (5) confer or modify other physicochemical or functional properties of such analogs. Analogs can include various mutations of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure ©. Branden and J. Tooze, eds., Garland Publishing, New York, N.Y. (1991)); and Thornton et at. Nature 354:105 (1991), which are each incorporated herein by reference.

The term “polypeptide fragment” as used herein refers to a polypeptide that has an amino-terminal and/or carboxy-terminal deletion, but where the remaining amino acid sequence is identical to the corresponding positions in the naturally-occurring sequence deduced, for example, from a full-length cDNA sequence. Fragments typically are at least 5, 6, 8 or 10 amino acids long, such as at least 14 amino acids long or at least 20 amino acids long, usually at least 50 amino acids long or at least 70 amino acids long.

“Antibody” or “antibody peptide(s)” refer to an intact antibody, or a binding fragment thereof that competes with the intact antibody for specific binding. Binding fragments are produced by recombinant DNA techniques, or by enzymatic or chemical cleavage of intact antibodies. Binding fragments include Fab, Fab′, F(ab′)₂, Fv, and single-chain antibodies. An antibody other than a “bispecific” or “bifunctional” antibody is understood to have each of its binding sites identical. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay).

The term “MHC” as used herein will be understood to refer to the Major Histocompability Complex, which is defined as a set of gene loci specifying major histocompatibility antigens. The term “HLA” as used herein will be understood to refer to Human Leukocyte Antigens, which is defined as the histocompatibility antigens found in humans. As used herein, “HLA” is the human form of “MHC”.

The terms “MHC light chain” and “MHC heavy chain” as used herein will be understood to refer to portions of the MHC molecule. Structurally, class I molecules are heterodimers comprised of two noncovalently bound polypeptide chains, a larger “heavy” chain (α) and a smaller “light” chain (β-2-microglobulin or β2m). The polymorphic, polygenic heavy chain (45 kDa), encoded within the MHC on chromosome six, is subdivided into three extracellular domains (designated 1, 2, and 3), one intracellular domain, and one transmembrane domain. The two outermost extracellular domains, 1 and 2, together form the groove that binds antigenic peptide. Thus, interaction with the TCR occurs at this region of the protein. The 3 domain of the molecule contains the recognition site for the CD8 protein on the CTL; this interaction serves to stabilize the contact between the T cell and the APC. The invariant light chain (12 kDa), encoded outside the MHC on chromosome 15, consists of a single, extracellular polypeptide. The terms “MHC light chain”, “β-2-microglobulin”, and “β2m” may be used interchangeably herein.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. An antibody is said to specifically bind an antigen when the dissociation constant is <1 μM, or <100 nM, or <10 nM.

The term “antibody” is used in the broadest sense, and specifically covers monoclonal antibodies (including full length monoclonal antibodies), polyclonal antibodies, multispecific antibodies (e.g., bispecific antibodies), and antibody fragments (e.g., Fab, F(ab′)₂ and Fv) so long as they exhibit the desired biological activity. Antibodies (Abs) and immunoglobulins (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

Native antibodies and immunoglobulins are usually heterotetrameric glycoproteins of about 150,000 daltons, composed of two identical light (L) chains and two identical heavy (H) chains. Each light chain is linked to a heavy chain by one covalent disulfide bond. While the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., J. Mol. Biol. 186, 651-66, 1985); Novotny and Haber, Proc. Natl. Acad. Sci. USA 82 4592-4596 (1985).

An “isolated” antibody is one which has been identified and separated and/or recovered from a component of the environment in which is was produced. Contaminant components of its production environment are materials which would interfere with diagnostic or therapeutic uses for the antibody, and may include enzymes, hormones, and other proteinaceous or nonproteinaceous solutes. In certain embodiments, the antibody will be purified as measurable by at least three different methods: 1) to greater than 50% by weight of antibody as determined by the Lowry method, such as more than 75% by weight, or more than 85% by weight, or more than 95% by weight, or more than 99% by weight; 2) to a degree sufficient to obtain at least 10 residues of N-terminal or internal amino acid sequence by use of a spinning cup sequentator, such as at least 15 residues of sequence; or 3) to homogeneity by SDS-PAGE under reducing or non-reducing conditions using Coomasie blue or, preferably, silver stain. Isolated antibody includes the antibody in situ within recombinant cells since at least one component of the antibody's natural environment will not be present. Ordinarily, however, isolated antibody will be prepared by at least one purification step.

The term “antibody mutant” refers to an amino acid sequence variant of an antibody wherein one or more of the amino acid residues have been modified. Such mutants necessarily have less than 100% sequence identity or similarity with the amino acid sequence having at least 75% amino acid sequence identity or similarity with the amino acid sequence of either the heavy or light chain variable domain of the antibody, such as at least 80%, or at least 85%, or at least 90%, or at least 95%.

The term “variable” in the context of variable domain of antibodies, refers to the fact that certain portions of the variable domains differ extensively in sequence among antibodies and are used in the binding and specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains. There are at least two techniques for determining CDRs: (1) an approach based on cross-species sequence variability (i.e., Kabat et al., Sequences of Proteins of Immunological Interest (National Institute of Health, Bethesda, Md. 1987); and (2) an approach based on crystallographic studies of antigen-antibody complexes (Chothia, C. et al. (1989), Nature 342: 877). The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies (see Kabat et al.) The constant domains are not involved directly in binding an antibody to an antigen, but exhibit various effector function, such as participation of the antibody in antibody-dependent cellular toxicity.

The term “antibody fragment” refers to a portion of a full-length antibody, generally the antigen binding or variable region. Examples of antibody fragments include Fab, Fab′, F(ab′)₂ and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments which are capable of cross-linking antigen, and a residual other fragment (which is termed pFc′). As used herein, “functional fragment” with respect to antibodies, refers to Fv, F(ab) and F(ab′)₂ fragments.

An “Fv” fragment is the minimum antibody fragment which contains a complete antigen recognition and binding site. This region consists of a dimer of one heavy and one light chain variable domain in a tight, non-covalent association (V_{H}—V_{L} dimer). It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

The Fab fragment [also designated as F(ab)] also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains have a free thiol group. F(ab′) fragments are produced by cleavage of the disulfide bond at the hinge cysteines of the F(ab′)₂ pepsin digestion product. Additional chemical couplings of antibody fragments are known to those of ordinary skill in the art.

The light chains of antibodies (immunoglobulin) from any vertebrate species can be assigned to one of two clearly distinct types, called kappa (.kappa.) and lambda (.lambda.), based on the amino sequences of their constant domain.

Depending on the amino acid sequences of the constant domain of their heavy chains, “immunoglobulins” can be assigned to different classes. There are at least five (5) major classes of immunoglobulins: IgA, IgD, IgE, IgG and IgM, and several of these may be further divided into subclasses (isotypes), e.g., IgG-1, IgG-2, IgG-3 and IgG4; IgA-1 and IgA-2. The heavy chains constant domains that correspond to the different classes of immunoglobulins are called α, Δ, ε, γ and μ, respectively. The subunit structures and three-dimensional configurations of different classes of immunoglobulins are well known.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, Nature 256, 495 (1975), or may be made by recombinant methods, e.g., as described in U.S. Pat. No. 4,816,567. The monoclonal antibodies for use with the present invention may also be isolated from phage antibody libraries using the techniques described in Clackson et al. Nature 352: 624-628 (1991), as well as in Marks et al., J. Mol. Biol. 222: 581-597 (1991).

Utilization of the monoclonal antibodies of the present invention may require administration of such or similar monoclonal antibody to a subject, such as a human. However, when the monoclonal antibodies are produced in a non-human animal, such as a rodent, administration of such antibodies to a human patient will normally elicit an immune response, wherein the immune response is directed towards the antibodies themselves. Such reactions limit the duration and effectiveness of such a therapy. In order to overcome such problem, the monoclonal antibodies of the present invention can be “humanized”, that is, the antibodies are engineered such that antigenic portions thereof are removed and like portions of a human antibody are substituted therefor, while the antibodies' affinity for specific peptide/MHC complexes is retained. This engineering may only involve a few amino acids, or may include entire framework regions of the antibody, leaving only the complementarity determining regions of the antibody intact. Several methods of humanizing antibodies are known in the art and are disclosed in U.S. Pat. No. 6,180,370, issued to Queen et al on Jan. 30, 2001; U.S. Pat. No. 6,054,927, issued to Brickell on Apr. 25, 2000; U.S. Pat. No. 5,869,619, issued to Studnicka on Feb. 9, 1999; U.S. Pat. No. 5,861,155, issued to Lin on Jan. 19, 1999; U.S. Pat. No. 5,712,120, issued to Rodriquez et al on Jan. 27, 1998; and U.S. Pat. No. 4,816,567, issued to Cabilly et al on Mar. 28, 1989, the Specifications of which are all hereby expressly incorporated herein by reference in their entirety.

Humanized forms of antibodies are chimeric immunoglobulins, immunoglobulin chains or fragments thereof (such as Fv, Fab, Fab′, F(ab′)2 or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., 1986; Riechmann et al., 1988; Verhoeyen et al., 1988), by substituting rodent CDRs or CDR sequences for the corresponding sequences of a human antibody. (See also U.S. Pat. No. 5,225,539.) In some instances, Fᵥ framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, 1992).

97 published articles relating to the generation or use of humanized antibodies were identified by a PubMed search of the database as of Apr. 25, 2002. Many of these studies teach useful examples of protocols that can be utilized with the present invention, such as Sandborn et al., Gatroenterology, 120:1330 (2001); Mihara et al., Clin. Immunol. 98:319 (2001); Yenari et al., Neurol. Res. 23:72 (2001); Morales et al., Nucl. Med. Biol. 27:199 (2000); Richards et al., Cancer Res. 59:2096 (1999); Yenari et al., Exp. Neurol. 153:223 (1998); and Shinkura et al., Anticancer Res. 18:1217 (1998), all of which are expressly incorporated in their entirety by reference. For example, a treatment protocol that can be utilized in such a method includes a single dose, generally administered intravenously, of 10-20 mg of humanized mAb per kg (Sandborn, et al. 2001). In some cases, alternative dosing patterns may be appropriate, such as the use of three infusions, administered once every two weeks, of 800 to 1600 mg or even higher amounts of humanized mAb (Richards et al., 1999). However, it is to be understood that the invention is not limited to the treatment protocols described above, and other treatment protocols which are known to a person of ordinary skill in the art may be utilized in the methods of the present invention.

The presently disclosed and claimed embodiments further include fully human monoclonal antibodies against specific peptide/MHC complexes. Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., Hybridoma, 2:7 (1983)) and the EBV hybridoma technique to produce human monoclonal antibodies (see Cole, et al., PNAS 82:859 (1985)). Human monoclonal antibodies may be utilized in the practice of the present invention and may be produced by using human hybridomas (see Cote, et al., PNAS 80:2026 (1983)) or by transforming human B-cells with Epstein Barr Virus in vitro (see Cole, et al., 1985).

In addition, human antibodies can also be produced using additional techniques, including phage display libraries (Hoogenboom et al., Nucleic Acids Res. 19:4133 (1991); Marks et al., J Mol Biol. 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al., J Biol. Chem. 267:16007 (1992); Lonberg et al., Nature, 368:856 (1994); Morrison, 1994; Fishwild et al., Nature Biotechnol. 14:845 (1996); Neuberger, Nat. Biotechnol. 14:826 (1996); and Lonberg and Huszar, Int Rev Immunol. 13:65 (1995).

Human antibodies may additionally be produced using transgenic nonhuman animals which are modified so as to produce fully human antibodies rather than the animal's endogenous antibodies in response to challenge by an antigen. (See PCT publication WO 94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. One embodiment of such a nonhuman animal is a mouse, and is termed the XENOMOUSE™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598, issued to Kucherlapati et al. on Aug. 17, 1999, and incorporated herein by reference. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771, issued to Hori et al. on Jun. 29, 1999, and incorporated herein by reference. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

The term “agent” is used herein to denote a chemical compound, a mixture of chemical compounds, a biological macromolecule, or an extract made from biological materials.

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance.

The term “pharmaceutical agent or drug” as used herein refers to a chemical compound or composition capable of inducing a desired therapeutic effect when properly administered to a patient. Other chemistry terms herein are used according to conventional usage in the art, as exemplified by The McGraw-Hill Dictionary of Chemical Terms (Parker, S., Ed., McGraw-Hill, San Francisco (1985)), incorporated herein by reference).

The term “antineoplastic agent” is used herein to refer to agents that have the functional property of inhibiting a development or progression of a neoplasm in a human, particularly a malignant (cancerous) lesion, such as a carcinoma, sarcoma, lymphoma, or leukemia. Inhibition of metastasis is frequently a property of antineoplastic agents.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, such as more than about 85%, 90%, 95%, and 99%. In one embodiment, the object species is purified to essential homogeneity (contaminant species cannot be detected in the composition by conventional detection methods) wherein the composition consists essentially of a single macromolecular species.

The term patient includes human and veterinary subjects.

A “liposome” is a small vesicle composed of various types of lipids, phospholipids and/or surfactant. The components of the liposome are commonly arranged in a bilayer formation, similar to the lipid arrangement of biological membranes.

“Treatment” refers to both therapeutic treatment and prophylactic or preventative measures. Those in need of treatment include those already with the disorder as well as those in which the disorder is to be prevented.

A “disorder” is any condition that would benefit from treatment with the polypeptide. This includes chronic and acute disorders or diseases including those pathological conditions which predispose the mammal to the disorder in question.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include squamous cell cancer, small-cell lung cancer, non-small cell lung cancer, gastrointestinal cancer, pancreatic cancer, glioblastoma, cervical cancer, ovarian cancer, liver cancer, bladder cancer, hopatoma, breast cancer, colon cancer, colorectal cancer, endometrial carcinoma, salivary gland carcinoma, kidney cancer, renal cancer, prostate cancer, vulval cancer, thyroid cancer, hepatic carcinoma and various types of head and neck cancer.

“Mammal” for purposes of treatment refers to any animal classified as a mammal, including human, domestic and farm animals, nonhuman primates, and zoo, sports, or pet animals, such as dogs, horses, cats, cows, etc.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine and lysine.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition it is understood that, in any compound described herein having one or more double bond(s) generating geometrical isomers that can be defined as E or Z, each double bond may independently be E or Z a mixture thereof.

Likewise, it is understood that, in any compound described, all tautomeric forms are also intended to be included. For example all tautomers of phosphate groups are intended to be included. Furthermore, all tautomers of heterocyclic bases known in the art are intended to be included, including tautomers of natural and non-natural purine-bases and pyrimidine-bases.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the methods and combinations described herein include crystalline forms (also known as polymorphs, which include the different crystal packing arrangements of the same elemental composition of a compound), amorphous phases, salts, solvates, and hydrates. In some embodiments, the compounds described herein exist in solvated forms with pharmaceutically acceptable solvents such as water, ethanol, or the like.

In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

What is claimed is:

1. A composition comprising a monoclonal antibody or biologic configured to bind in a ligand independent manner to IL7R, for use in the treatment of acute lymphoblastic leukemia.

2. The composition of claim 1, for use in the treatment of Philadelphia chromosome positive acute lymphoblastic leukemia.

3. The composition of claim 1, for use in the treatment of Resistant Philadelphia chromosome positive acute lymphoblastic leukemia.

4. The composition of claim 1, comprising a monoclonal antibody.

5. The composition of claim 1, wherein the monoclonal antibody or biologic is configured to bind to the IL7R common gamma chain.

6. The composition of claim 1, wherein the monoclonal antibody is a fragment of anti-IL7R antibody.

7. The composition of claim 1, wherein the monoclonal antibody is selected from the group consisting of murine, human, or humanized monoclonal antibody, and wherein the antibody is configured to bind to human IL-7R.

8. The composition of claim 1, wherein the monoclonal antibody is only the antibody or conjugate to the antibody or conjugate to a fragment of anti-IL7R antibody.

9. The composition of claim 1, wherein the monoclonal antibody comprises human framework regions, optionally for use in CAR-T-Cell immunotherapy.

10. The composition of claim 1, wherein the monoclonal antibody comprises at least one human constant region, and wherein the at least one human constant region comprises a modification that increases binding to the Philadelphia chromosome positive in IL7R.

11. The composition of claim 1, wherein the monoclonal antibody inhibits IL7 signaling in IL7R Philadelphia chromosome positive cells.

12. The composition of claim 1, wherein the monoclonal antibody or biologics specifically binds to or target the extracellular domain of the IL7R.

13. The composition of claim 1, wherein the monoclonal antibody comprises an anti-IL7R antibody that shows selectivity in targeting Philadelphia chromosome-positive IL7R antibodies relative to Philadelphia chromosome-negative IL7R antibodies, optionally for use CAR-T-Cell immunotherapy.

14. An antibody therapeutics composition adapted for administration by injection, comprising a therapeutically effective amount of the composition of claim 1 and a pharmaceutically acceptable excipient.

15. The antibody therapeutics composition of claim 14, further comprising an effector molecule or a detectable marker wherein the monoclonal antibody or antigen binding fragment is linked to the effector molecule or the detectable marker.

16. The antibody therapeutics composition of claim 14, further comprising a therapeutic drug, wherein the therapeutic drug and the monoclonal antibody form an antibody-drug conjugate.

17. The antibody therapeutics composition of claim 16, wherein the therapeutic drug is a chemotherapeutic agent for the treatment of acute lymphoblastic leukemia or chronic lymphocytic leukemia.

18. The antibody therapeutics composition of claim 16, further comprising a cleavable peptide linker molecule, wherein the therapeutic drug is conjugated to the monoclonal antibody by the cleavable peptide linker.

19. A method for treatment of acute lymphoblastic leukemia comprising:
administering a therapeutically effective amount of the monoclonal antibody or biologics of claim 1 to a patient in need thereof for the treatment of Philadelphia chromosome positive acute lymphoblastic leukemia.

20. The method of claim 19, wherein the Philadelphia chromosome positive acute lymphoblastic leukemia is resistant to treatment with ABL kinase inhibitors.
